(19) United States
(12) Patent Application Publication
(10) Pub. No.: US 2006/0224405 A1
(43) Pub. Date: Oct. 5, 2006

(54) System and method for completing treatment authorization request forms

(76) Inventors: Amanda E. White, Schaumburg, IL (US); Karen D. Egan, Arlington Heights, IL (US); Charles Goodall, Hawthorn Woods, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/099,165

(22) Filed: Apr. 5, 2005

(51) Int. Cl. G06Q 99/00

(52) U.S. Cl. 705/1

(57) ABSTRACT

A system and method are provided for completing treatment authorization request forms that includes receiving a pharmaceutical prescription's data, choosing a treatment authorization request form, associating the form's fields with the prescription data, and merging the data and the form into an integrated file where the prescription data is represented on the form's fields.

### TECHNICAL FIELD

The present patent relates generally to techniques for facilitating the completion of Treatment Authorization Request (TAR) forms, and more particularly to automatically populating the form's fields upon the rejection of a prescription request and associating the prescription data with that form.

### BACKGROUND

Many pharmacies rely in large part on the business generated by customers enrolled in managed health care organizations. Often, these organizations place restrictions on the number of prescriptions their members may fill without specific approval. When customers present prescriptions requiring a secondary approval, the administrative authorization process can often cause delay. Specifically, once the user enters the customer and prescription data, a request needing further approval will generate a rejection. Upon rejection, the user will have to manually re-enter all customer and prescription data onto a paper treatment authorization request form and transmit this form to the approving agency. Manually filling out each form, especially after the user previously entered the same data, is time-consuming and prone to error. As a result, a system is needed to reduce or eliminate manually completing treatment authorization request forms which saves time and reduces the likelihood of error.

### BRIEF DESCRIPTION OF THE DRAWINGS

FIG. 1 is a block diagram of an embodiment of an intelligent network system.

FIG. 2 is a block diagram of an alternative embodiment of an intelligent network system that includes a Treatment Authorization Request Form database.

FIG. 3 is a block diagram of an alternative embodiment of an intelligent network system that includes a connected client device.

FIG. 4 is a schematic diagram of some of the components of the network server shown in FIGS. 1, 2, and 3.

FIG. 5 is a schematic diagram of an embodiment of one of the facilities shown schematically in FIGS. 1, 2, and 3.

FIG. 6 is a flowchart showing some of the steps used to facilitate the completion of Treatment Authorization Request Forms.

FIG. 7 a flowchart showing some of the steps used in an alternative embodiment to the embodiment shown in FIG. 6 and includes barcode technology.

### DETAILED DESCRIPTION OF VARIOUS EMBODIMENTS

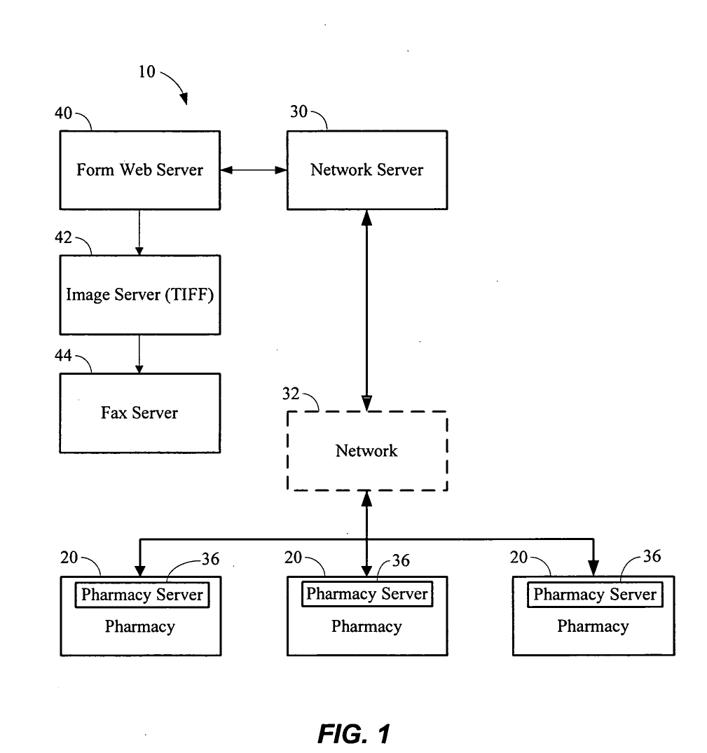

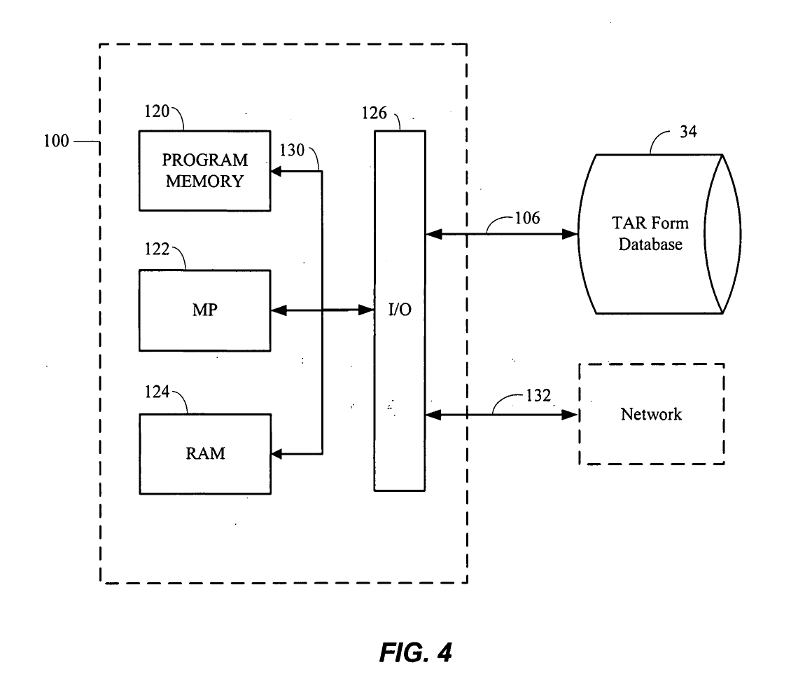

FIG. 1 illustrates an embodiment of a data network 10 that may be used to facilitate the completion of Treatment Authorization Request Forms for a large number of prescription fills that occur throughout several pharmacies that are each located in different geographic locations. Referring to FIG. 1, the data network 10 may include a first group of pharmacies or facilities 20 operatively coupled to a network server or machine 30 via a network 32. The network server or machine 30 may also include or may be connected to a Treatment Authorization Request Form database, discussed in greater detail below with reference to FIG. 4. The plurality of pharmacies 20 may be located, by way of example rather than limitation, in separate geographic locations from each other, in different areas of the same city, or in different states. Also, the pharmacies 20 may be affiliated with a single entity or with multiple entities. The pharmacies 20 may be located in a conventional retail store or they may be located in proximity with a drug warehouse or distribution center that would include a shipping facility and would not be accessible to the general public.

The network 32 may be provided using a wide variety of techniques well known to those skilled in the art for the transfer of electronic data. For example, the network 32 may comprise dedicated access lines, plain, ordinary telephone lines, satellite links, combinations of these, etc. Additionally, the network 32 may include a plurality of network computers or server computers (not shown), each of which may be operatively interconnected in a known manner. Where the network 32 comprises the Internet, data communication may take place over the network 32 via an Internet communication protocol.

The network server 30 may be a server computer of the type commonly employed in networking solutions, and it may also have a data structure into which customer account data and prescription fill data is retained. The network server 30 may be used to accumulate, analyze, and download data relating to the operation of the pharmacies 20 and more particularly to information pertaining to Treatment Authorization Request Forms, insurance companies, federal and state authorization agencies, and rejected prescriptions that have been submitted on Treatment Authorization Request Forms at the pharmacies 20 or another central or regional location.

For example, the network server 30 may periodically receive data from each of the pharmacies 20 indicative of prescriptions that have been attempted to be filled but require submission on Treatment Authorization Request Forms as well as rejected Treatment Authorization Request Forms. This information may be accumulated and periodically transferred to an off-site facility for purposes of data storage, report generation, etc. The information may alternatively be purged from storage on a periodic basis.

The pharmacies 20 may include one or more pharmacy servers 36 that may be utilized to store information on drugs, the weights of those drugs, and other information pertaining to those drugs. For example, the pharmacy servers 36 may store information pertaining to Treatment Authorization Request Forms, insurance companies, federal and state authorization agencies, and rejected prescriptions that have been submitted on Treatment Authorization Request Forms at the pharmacies 20 or another central or regional location. The pharmacy servers 36 may also store information related to prescriptions filled at the pharmacy 36 that may be used to generate a wide variety of reports, such as, for example, error reports, approval reports, override reports, etc.

The data network 10 may also include a Form Web Server 40 operatively coupled to the network server 30. The data network 10 may also include an image server 42 operatively coupled to the form web server 40 and a fax server 44 operatively coupled to the image server 42. Those of ordinary skill in the art will readily appreciate that many alternative couplings could also be contemplated.

The pharmacy servers 36 may store and run an application that automatically populates a Treatment Authorization Request Form's fields upon the rejection of a prescription request. The application may also associate a set of prescription data and a control number with the particular Treatment Authorization Request Form. Alternatively, the application could be stored and run on the form web server 40 or a combination of the form web server 40 and the pharmacy server 36. The application could be configured to allow a proactive submission of a prescription request on a Treatment Authorization Request Form. The fax server 44 could be utilized for a fax retry cycle to place a failed fax into a fax retry pool for a later fax attempt during a subsequent cycle, as well as sending notification, such as an email, back to one or more of the pharmacies 36. These actions are described in greater detail below with reference to the flowcharts.

Although the data network 10 is shown to include one network server 30 and three pharmacies 20, it should be understood that different numbers of computers and pharmacies may be utilized. For example, the network 32 may include a plurality of network servers 30 and hundreds or thousands of pharmacies 20, all of which may be interconnected via the network 32. According to the disclosed example, this configuration may provide several advantages, such as, for example, enabling near real time uploads and downloads of information as well as periodic uploads and downloads of information. This provides for a primary backup of all the information generated in transactions where prescriptions are filled and rejected and where Treatment Authorization Request Forms are completed and submitted, as well as the status of the prescriptions associated with the submitted forms.

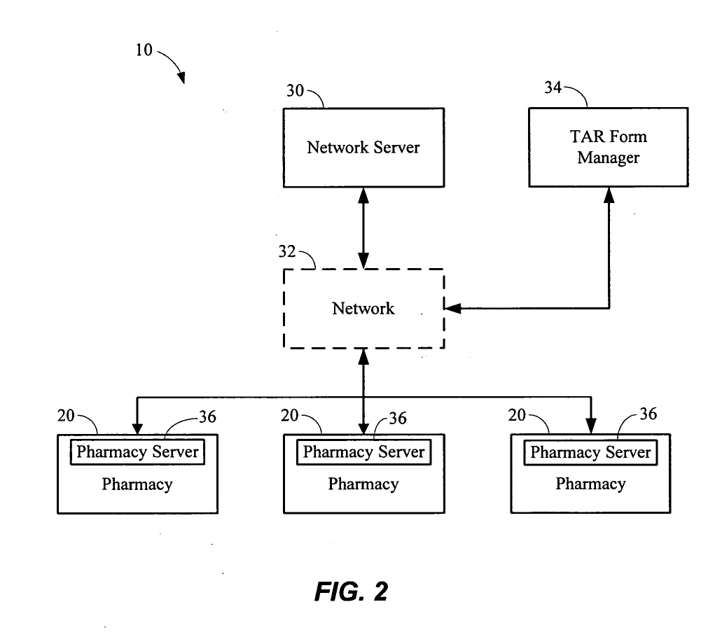

FIG. 2 illustrates an alternative embodiment of the network 10 shown in FIG. 1, wherein a central Treatment Authorization Request Form manager 34 is used to manage the Treatment Authorization Request Forms that are submitted for rejected prescriptions as well as their approvals and rejections. The Treatment Authorization Request Form manager 34 may also be used as storage for future Treatment Authorization Request Forms to be used by the pharmacies 20. The central Treatment Authorization Request Form manager 34 is shown separately in FIG. 2, but could be a functional entity implemented on the network server 30 as shown in FIG. 1, or elsewhere. The embodiment of FIG. 2 is similar to the embodiment shown in FIG. 1 and includes many of the same structures and components. For clarity, the structures and components remaining the same are shown with like reference numbers as those from FIG. 1. Referring to FIG. 2, the Treatment Authorization Request Form manager 34 may be linked to the network 32 so that data may be transferred between the Treatment Authorization Request Form manager 34 and the network server 30 and the pharmacies 20.

The Treatment Authorization Request Form manager 34 may be used as a repository to store information pertaining to Treatment Authorization Request Forms for various states, or other third party payers or prescribers, prescriptions submitted on Treatment Authorization Request Forms and their current status, and other data corresponding to prescriptions filled and attempted to be filled at the pharmacies 20. As with the network server 30 in FIG. 1, the Treatment Authorization Request Form manager 34 may periodically receive data from each of the pharmacies 20 indicative of prescriptions submitted on Treatment Authorization Request Forms and their status. The Treatment Authorization Request Form manager 34 may be an unrelated third party, or it may be a subsidiary or division of the owner of the pharmacies.

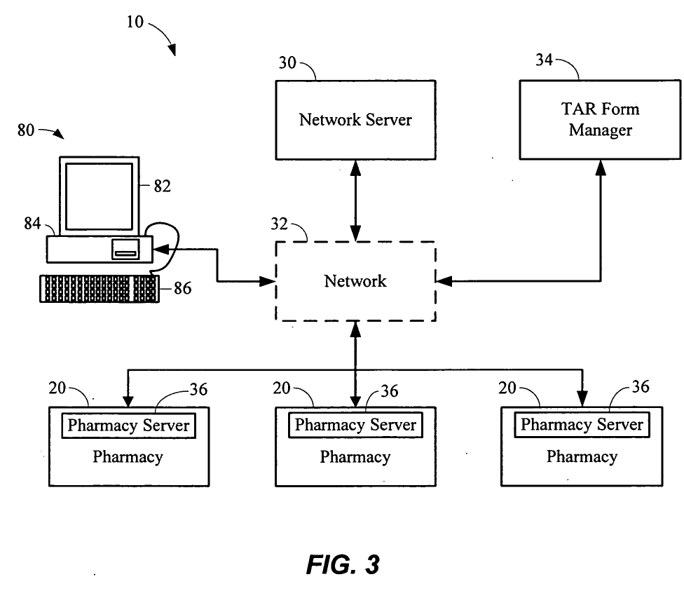

FIG. 3 illustrates an alternative embodiment of the network 10 shown in FIG. 1, wherein a client device 80 is linked to the network 32 to enable a customer to order a prescription to be filled using the client device 80. The embodiment of FIG. 3 is similar to the embodiment shown in FIGS. 1 and 2 and includes many of the same structures and components. For clarity, the structures and components remaining the same are shown with like reference numbers as those from FIGS. 1 and 2.

Referring to FIG. 3, the client device 80 may be any suitable device for accessing the network 32, such as a computer, PDA, web enabled cell phone, etc., and is shown to include a display 82, a controller 84, a keyboard 86, as well as a variety of other input/output devices. The client device 80 may be linked to the network 32 so that a customer may order a prescription to be filled without having to physically visit one of the pharmacies 20. Thus, it could be the patient, or a person associated with the patient, that initiates the process. Access may be provided, for example, over the Internet with a website that is associated with the pharmacies 20.

The client device 80 may allow a customer to submit a Treatment Authorization Request Form for a prescription that was previously rejected, without having to physically visit one of the pharmacies 20. The pharmacy organization may provide the customer the option of having the prescription drug(s) shipped to the customer or having the prescription drug(s) made available at a local (or any other) retail pharmacy 20 for pickup by the customer.

While the network 10 is shown to include one network server 30, one Treatment Authorization Request Form manager 34, three pharmacies 20, and one client device 80, it should be understood that different-numbers of computers, pharmacies, and client devices may be utilized. For example, the network 32 may include a plurality of network servers 30, a plurality of Treatment Authorization Request Form managers 34 and or databases, hundreds or thousands of pharmacies 20, and a plurality of client devices 80, all of which may be interconnected via the network 32.

FIG. 4 is a schematic diagram of one possible embodiment of the network server 30 shown in FIGS. 1, 2, and 3. The network server 30 may have a controller 100 that is operatively connected to a Treatment Authorization Request Form database 102 via a link 106. It should be noted that, while not shown, additional databases may be linked to the controller 100 in a known manner.

The controller 100 may include a program memory 120, a microcontroller or a microprocessor (MP) 122, a random-access memory (RAM) 124, and an input/output (I/O) circuit 126, all of which may be interconnected via an address/data bus 130. It should be appreciated that although only one microprocessor 122 is shown, the controller 100 may include multiple microprocessors 122. Similarly, the memory of the controller 100 may include multiple RAMs 124 and multiple program memories 120. Although the I/O circuit 126 is shown as a single block, it should be appreciated that the I/O circuit 126 may include a number of different types of I/O circuits. The RAM(s) 124 and programs memories 120 may be implemented as semiconductor memories, magnetically readable memories, and/or optically readable memories, for example. All of these memories or data repositories may be referred to as machine accessible mediums. The controller 100 may also be operatively connected to the network 32 via a link 130.

For the purpose of this description and as briefly discussed above, a machine-accessible medium includes any mechanism that provides (i.e., stores and/or transmits) information in a form accessible by a machine (e.g., a computer, network device, personal digital assistant, manufacturing tool, any device with a set of one or more processors). For example, a machine-accessible medium includes recordable/non-recordable media (e.g., read only memory (ROM); random access memory (RAM); magnetic disk storage media; optical storage media; flash memory devices), as well as electrical, optical, acoustical or other form of propagated signals (e.g., carrier waves, infrared signals, digital signals); etc.

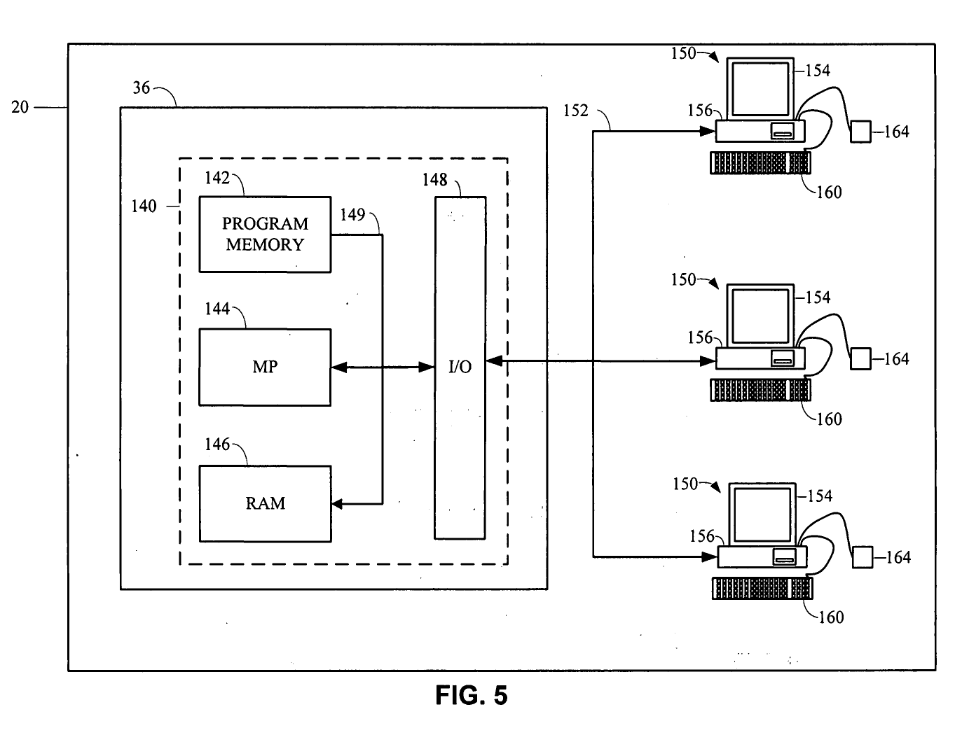

FIG. 5 is a schematic diagram of one possible embodiment of several components located in one or more of the pharmacies 20 from FIGS. 1, 2, and 3. Although the following description addresses the design of the pharmacies 20, it should be understood that the design of one or more of the pharmacies 20 may be different than the design of other pharmacies 20. Also, each pharmacy 20 may have various different structures and methods of operation. It should also be understood that the embodiment shown in FIG. 5 illustrates some of the components and data connections present in a pharmacy 20, however it does not illustrate all of the data connections present in a typical retail store in which the pharmacy is part of (i.e., a photo department, a cosmetic department, a plurality of front line terminals, etc.). For exemplary purposes, various designs of the pharmacies are described below, but it should be understood that numerous other designs may be utilized.

The pharmacy 20 may have a pharmacy server 36, which includes a controller 140, wherein the pharmacy server 36 is operatively connected to a plurality of workstations 150 via a network 152. The network 152 may be a wide area network (WAN), a local area network (LAN), or any other type of network readily known to those persons of ordinary skill in the art. The workstations 150 may also be operatively connected to the network server 30 as illustrated in FIGS. 1-3 via the network 32.

Similar to the controller 100 from FIG. 4, the controller 140 may include a program memory 142, a microcontroller or a microprocessor (MP) 144, a random-access memory (RAM) 146, and an input/output (I/O) circuit 148, all of which may be interconnected via an address/data bus 149. As discussed with reference to the controller 100, it should be appreciated that although only one microprocessor 144 is shown, the controller 140 may include multiple microprocessors 144. Similarly, the memory of the controller 140 may include multiple RAMs 146 and multiple programs memories 142. Although the I/O circuit 148 is shown as a single block, the I/O circuit 148 may include a number of different types of I/O circuits. The RAM(s) 146 and programs memories 142 may also be implemented as semiconductor memories, magnetically readable memories, and/or optically readable memories, for example.

The workstations 150 may include a display 154, a controller 156, a keyboard 160, a barcode reader 164 (either stationary or portable and either wired or wireless), as well as a variety of other input/output devices such as an RFID tag reader, a mouse, touch screen, track pad, track ball, isopoint, printer, card reader, voice recognition system, a keypad, a biometrics device (such as an iris reader or fingerprint scanner), etc. With regard to the barcode scanner 170, as discussed below, this device may be eliminated in some of the disclosed embodiments. Each workstation 150 may be signed onto and occupied by a pharmacy employee to assist them in performing their duties. Pharmacy employees may sign onto a workstation 150 using any generically available technique, such as entering a user name and password, using an ID card, or inputting biometric data. If a pharmacy employee is required to sign onto a workstation 150, this information may be passed via the link 152 to the pharmacy server 36 so that the controller 140 will be able to identify the signed on pharmacy employees and the corresponding workstation 150. This may be useful in monitoring the pharmacy employees' Treatment Authorization Request Form completion performance.

Typically, pharmacy servers 36 store a plurality of files, programs, and other data for use by the workstations 150 and the network server 30. One pharmacy server 36 may handle Treatment Authorization Request Forms from a large number of workstations 150. Accordingly, each pharmacy server 36 may typically comprise a high end computer with a large storage capacity, one or more fast microprocessors, and one or more high speed network connections. Conversely, relative to a typical pharmacy server 36, each workstation 150 may typically include less storage capacity, a single microprocessor, and a single network connection.

### Overall Operation of the System

One manner in which an exemplary system may operate is described below in connection with a number of flow charts which represent a number of portions or routines of one or more computer programs. As those of ordinary skill in the art will appreciate, the majority of the software utilized to implement the routines is stored in one or more of the memories in the controllers 100 and 140, and may be written at any high level language such as C, C++, C#, Java or the like, or any low-level assembly or machine language. By storing the computer program portions therein, various portions of the memories are physically and/or structurally configured in accordance with the computer program instructions. Parts of the software, however, may be stored and run locally on the terminals 150. As the precise location where the steps are executed can be varied without departing from the scope of the invention, the following figures do not address the machine performing an identified function.

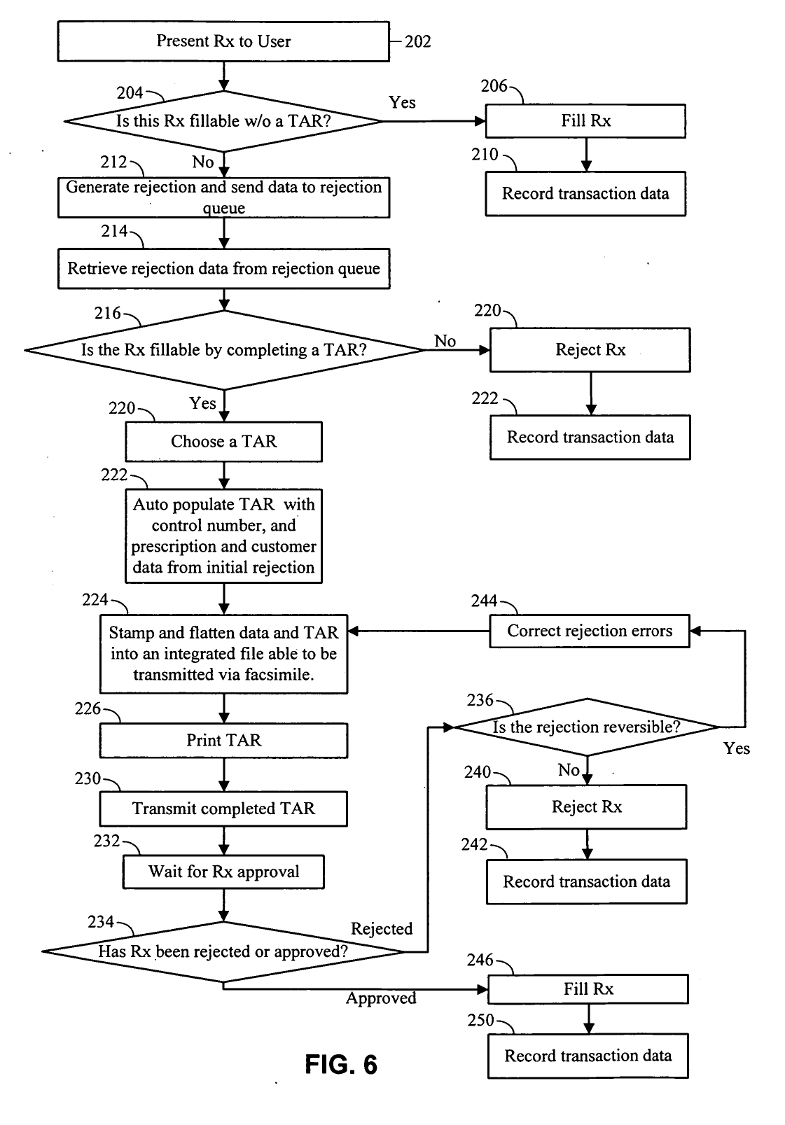

FIG. 6 is part of a flow chart 200 describing some of the steps used to automatically populate a Treatment Authorization Request: Form's fields upon the rejection of a prescription request. The flowchart 200 also describes the association of a set of prescription data and a control number with the particular Treatment Authorization Request Form. Some of the steps shown in the flowchart 200 may be stored in the memory of the controllers 100 and 140.

Referring to FIG. 6, the process flow 200 may begin when a customer presents a prescription to a pharmacy employee or other user (block 202). The prescription may include several pieces of data that define the particular prescription and the associated patient. The pieces of data may be referred to as elements. As a group, the pieces of data or the elements may form a data set.

In many situations a patient may be limited in the number of prescriptions that they may have filled in a particular time period by requirements established by the patient's healthcare insurance provider or a federal government agency such as Medicare. If the patient needs a prescription that exceeds the normal allotted number of prescriptions or a special prescription that requires special authorization, it may be necessary to fill out a Treatment Authorization Request Form. It should be noted that users may need to fill out an enrollment form for participation in the Treatment Authorization Request program as an initial matter.

As shown in the process flow 200, it may be determined if the prescription presented by the patient is fillable without a Treatment Authorization Request Form (block 204). If it is determined at the block 204 that the prescription is fillable without a Treatment Authorization Request Form, the pharmacy employee may fill the prescription (block 206) and the system may record the transaction data associated with the filled prescription in the program memory 120 or the Treatment Authorization Request Form database 34 or any other suitable memory (block 210).

If the pharmacy employee attempts to fill the prescription and it is determined at the block 204 that the prescription is not fillable without a Treatment Authorization Request Form, the rejection may be generated and a set of data associated with the rejection may be sent to a rejection que (block 212). It should also be noted that the prescription may be associated with a plurality of elements such as, for example, patient data and/or drug data.

The pharmacy may contact a prescriber associated with the prescription to obtain information for the Treatment Authorization Request Form. This information may include diagnosis description and medical justification for the prescription. Medicaid or any other authorizing agency may reject the Treatment Authorization Request if sufficient justification for the additional prescription request cannot be provided. Alternatively, prescribers may fill out a hard copy prescription pad or Treatment Authorization Request Form with the information on it, that includes specific services requested, and send this information with the patient to the pharmacy. A few examples of reasons for generating a rejection include: drug not covered, prior authorization needed, ingredient duplication, therapeutic duplication, invalid prescription denied, and coverage expired.

Once the prescription has been rejected, it may be retrieved from a rejection que (block 214). It may then be determined if the prescription is fillable by completing a Treatment Authorization Request Form (block 216). If the prescription is not fillable by completing the Treatment Authorization Request From, the prescription may be rejected (block 220), and the transaction data associated with the prescription attempted to be filled may be recorded in the program memory 120 or the Treatment Authorization Request Form database 102 or any other suitable memory (block 222).

Still referring to FIG. 6, the process flow 200 may continue with the selection of a Treatment Authorization Request Form (block 220). The selection for the Treatment Authorization Request Form may be performed automatically if there is only one state form that is to be used or if the system is adapted to identify an appropriate form based on information associated with the prescription, such as, for example, the patients home state of residence. Alternatively, the system may select an appropriate form based on criteria other than a state, such as an appropriate federal form or a benefit provider for the patient associated with the prescription.

A particular state may also be set as a default if more than one state Treatment Authorization Request Form is available and additional state Treatment Authorization Request Forms could be selected from a drop down menu. The selected Treatment Authorization Request Form may include a plurality of fields that correspond to at least a portion of the plurality of elements that may be associated with the prescription.

A Treatment Authorization Request control number may then be associated with the selected Treatment Authorization Request Form (block 222). The control number may be an alphanumeric code such as a sequence number. The control number may be given to the pharmacy by the authorizing agency in a predetermined fashion, or the control number may be automatically generated by the system and associated with the selected Treatment Authorization Request Form. At least a portion of the plurality of fields on the selected Treatment Authorization Request Form may be associated with at least a portion of the data set and the plurality of elements associated with the pharmaceutical prescription. In other words, the information associated with the data set and elements from the prescription is autopopulated into the plurality of fields on the Treatment Authorization Request Form.

Thus, the patient and prescription data and the Treatment Authorization Request Form are flattened and merged into an integrated file that is capable of being printed and transmitted such as, for example, via facsimile (block 226). The form can also be transmitted electronically as well, such as, for example, via email. It should be noted that before the data and the Treatment Authorization Request Form are merged, an image of the pharmacy manager's signature may be stamped on a signature line on the Treatment Authorization Request Form. Alternatively, a numeric authorization code, a digital signature, or any other electronic authorization could be applied to the completed Treatment Authorization Request Form to indicate an appropriate level of approval. Any form that requires the patient's signature could incorporate an electronic signature that was captured/submitted via the client device 80.

After the completed form has been stamped and flattened (converted to a stand alone file) it may be converted to a different format, such as, for example TIFF or PDF. The merged form may then be printed (block 224) and/or displayed for the pharmacy employee. The pharmacy employee or other such person associated with the pharmacy may then transmit the completed Treatment Authorization Request Form having the data associated with the patient and the prescription autopopulated and merged into the form to the appropriate authorizing party (block 230). The merged form would not necessarily need to be printed. The merged form could be transmitted directly from the electronic copy on the computer by facsimile or e-mail or any other electronic form of transmission. For example, the merged form could be converted to a PDF file for attachment to an e-mail message or transmission via facsimile without ever being printed.

Once the completed Treatment Authorization Request Form has been transmitted to the authorizing party, the completed form may be stored in a memory such as the program memory 120 or the Treatment Authorization Request Form database 34, or any other suitable memory. The pharmacy may then wait for the approval of the prescription from the authorizing party (block 232).

Once notification has been received from the authorizing party regarding the completed Treatment Authorization Request Form, it may be determined if the prescription has been rejected or approved (blocked 234). If it is determined at block 234 that the prescription has been rejected, it may be determined if the rejection is reversible (block 236). If it is determined that the prescription is rejected for a reason that is not reversible, the prescription will be rejected (block 240) and the transaction data associated with the rejection of the submitted prescription will be recorded in an appropriate memory (block 242). If it is determined at the block 236 that the prescription was rejected for a reversible reason, the user may edit the completed Treatment Authorization Request Form to correct the rejection errors (block 244). Thereafter, the process flow returns to stamp and flatten the modified data and the Treatment Authorization Request Form for a subsequent transmission to the authorizing party.

If it was determined at the block 234 that the prescription is approved by the authorizing party, the prescription may be filled (block 246) and the transaction data associated with the correct prescription may be recorded in an appropriate memory (block 250). It should also be noted that the completed TAR forms, whether approved or rejected, may be stored in a memory such as the Treatment Authorization Request Form database 34 for an indefinite period of time or for a predetermined period of time to facilitate refills for the prescription. In addition to the data set elements associated with the prescription that is associated with the plurality of elements from the Treatment Authorization Request Form, the system may also automatically populate an NDC-UPC field on a Treatment Authorization Request Form that is based on the drug name associated with the prescription.

The invention has been described in terms of several preferred embodiments. It will be appreciated that the invention may otherwise be embodied without departing from the fair scope of the invention defined by the following claims.

What is claimed is:

1. A method of providing field autopopulation for a treatment authorization request form comprising:
receiving a pharmaceutical prescription, the prescription being associated with a data set, the data set having a plurality of elements;
identifying a treatment authorization request form, the treatment authorization request form having a plurality of fields, at least a portion of the plurality of fields corresponding to at least a portion of the plurality of elements;
associating the plurality of fields with the data set;
determining if a control number is required for the treatment authorization request form;
associating the control number with the treatment authorization request form if it was determined that the control number was required; and
merging the data set, the control number, and the treatment authorization request form into an integrated file, at least a portion of the plurality of elements and the control number being represented on at least a portion of the plurality of fields.

2. The method of claim 1, further comprising prompting a user to input the control number.

3. The method of claim 1, further comprising automatically generating the control number.

4. The method of claim 1, wherein the control number is an alphanumeric sequence number.

5. The method of claim 1, wherein identifying a treatment authorization request form comprises choosing an appropriate state treatment authorization request form corresponding to a state in which the pharmaceutical prescription is filled.

6. The method of claim 1, wherein identifying a treatment authorization request form comprises choosing an appropriate treatment authorization request form based on a benefit provider associated with the pharmaceutical prescription.

7. The method of claim 1, further comprising displaying the treatment authorization request form with the data set, at least a portion of the plurality of elements being aligned with at least a portion of the plurality of fields.

8. The method of claim 1, further comprising printing the treatment authorization request form with the data set and the control number with at least a portion of the plurality of elements and the control number aligned with at least a portion of the plurality of fields.

9. The method of claim 1, further comprising transmitting the treatment authorization request form with the data set and the control number aligned with at least a portion of the plurality of fields.

10. The method of claim 1, further comprising providing the ability to modify the plurality of elements.

11. The method of claim 10, further comprising correcting a rejection from an authorizing agency, wherein the rejection is due to reversible errors.

12. The method of claim 10, further comprising receiving the control number from an authorizing agency.

13. A method of providing field autopopulation for a treatment authorization request form comprising:
receiving a pharmaceutical prescription, the prescription being associated with a plurality of elements;
attempting to fulfill the prescription;
generating a rejection if the attempted prescription fill was rejected;
sending the rejection to a designated data structure where the rejection maintains an association with the plurality of elements;
identifying a treatment authorization request form, the treatment authorization request form having a plurality of fields corresponding to at least a portion of the plurality of elements;
associating at least a portion of the plurality of fields with at least a portion of the plurality of elements;
merging the plurality of elements and the treatment authorization request form into an integrated file, at least a portion of the plurality of elements being represented on at least a portion of the plurality of fields; and
transmitting the integrated file to an authorizing party.

14. The method of claim 13, further comprising associating a control number with the treatment authorization request-form.

15. The method of claim 14, further comprising prompting a user to input the control number.

16. The method of claim 14, further comprising automatically generating the control number.

17. The method of claim 14, wherein the control number is an alphanumeric sequence number.

18. The method of claim 13, wherein identifying a treatment authorization request form comprises choosing an appropriate state treatment authorization request form corresponding to a state in which the pharmaceutical prescription is filled.

19. The method of claim 13, further comprising generating a rejection based on an attempt to fulfill the prescription.

20. The method of claim 13, further comprising displaying the treatment authorization request form with at least a portion of the plurality of elements being aligned with at least the portion of the plurality of fields.

21. The method of claim 14, further comprising printing the treatment authorization request form with the control number and at least a portion of the plurality of elements and the control number aligned with the plurality of fields.

22. The method of claim 14, further comprising transmitting the treatment authorization request form with the control number aligned with at least a portion of the plurality of fields.

23. The method of claim 13, further comprising providing the ability to modify the plurality of elements.

24. The method of claim 23, further comprising correcting a rejection from an authorizing agency, wherein the rejection is due to reversible errors.

25. The method of claim 14, further comprising receiving the control number from the authorizing agency.

26. A treatment authorization request form field autopopulation system, comprising:
a first pharmacy at a first location, the first pharmacy having a workstation and a pharmacy server;
a second pharmacy at a second location, the second pharmacy having a workstation and a pharmacy server;
a network server and a network operatively coupling the network server and the workstations and the pharmacy servers at the first and second pharmacies;
the pharmacy server at the first pharmacy having a controller with a processor and a memory operatively coupled to the processor, the memory having a database to store information related to a prescription and a treatment authorization request form;
the workstation at the first pharmacy having a data input device to allow the prescription to be input into the system by a user, the prescription being associated with a data set, the data set having a plurality of elements;
the controller being programmed to:
receive periodic updates to the database from the network server;
transmit a request to fulfill the prescription;
receive a rejection in response to the request to fulfill the prescription;
send the rejection to a designated data structure wherein the rejection maintains an association with the data set;
associate a plurality of fields from a treatment authorization request form with the data set's elements;
associate a control number with the form; and
merge the data set, the control number, and the form into an integrated file, at least a portion of the data set's elements being represented on at least a portion of the form's fields.

27. The system of claim 26, wherein the controller is further programmed to prompt the user to input the control number.

28. The system of claim 26, wherein the controller is further programmed to generate the control number.

29. The system of claim 26, wherein the controller is further programmed to select an appropriate state treatment authorization request form corresponding to a state in which the prescription is to be filled, the state being one of the plurality of elements.

30. The system of claim 26, wherein the controller is further programmed to display the form with the data set, at least a portion of the plurality of elements being aligned with at least a portion of the plurality of fields.

31. The system of claim 26, wherein the controller is further programmed to print the form with the data set and the control number with at least a portion of the plurality of elements and the control number aligned with at least a portion of the plurality of fields.

32. The system of claim 26, wherein the controller is further programmed to allow the user to modify the plurality of elements.

33. The system of claim 26, wherein the controller is further programmed to allow the user to correct a second rejection, wherein the rejection is due to reversible errors.

34. The system of claim 26, wherein the controller is further programmed determine if the treatment authorization request form is needed to fill the prescription.

35. The system of claim 26, wherein the data input device is one of a barcode scanner, a radio frequency (RF) tag reader, a mouse, or a keyboard.

36. A treatment authorization request form field autopopulation system, comprising:
a first pharmacy at a first location, the first pharmacy having a workstation and a pharmacy server;
a second pharmacy at a second location, the second pharmacy having a workstation and a pharmacy server;
a network server and a network operatively coupling the network server and the workstations and the pharmacy servers at the first and second pharmacies;
the pharmacy server at the first pharmacy having a controller with a processor and a memory operatively coupled to the processor, the memory having a database to store information related to a prescription and a treatment authorization request form;
the workstation at the first pharmacy having a data input device to allow the prescription to be input into the system, the prescription being associated with a data set, the data set having a plurality of elements;
the controller being programmed to:
associate a plurality of fields from a treatment authorization request form with the data set's elements;
associate a control number with the form; and
merge the data set, the control number, and the form into an integrated file, at least a portion of the data set's elements being represented on at least a portion of the form's fields.

37. The system of claim 26, wherein the controller is further programmed to select an appropriate state treatment authorization request form corresponding to a state in which the prescription is to be filled, the state being one of the plurality of elements.

38. The system of claim 26, wherein the controller is further programmed to display the form with the data set, at least a portion of the plurality of elements being aligned with at least a portion of the plurality of fields.

39. The system of claim 26, wherein the controller is further programmed to print the form with the data set and the control number with at least a portion of the plurality of elements and the control number aligned with at least a portion of the plurality of fields.

40. The system of claim 26, wherein the controller is further programmed to allow the user to modify the plurality of elements.

41. A method of providing field autopopulation for a treatment authorization request form comprising:
receiving a pharmaceutical prescription, the prescription being associated with a data set, the data set having a plurality of elements;
identifying a treatment authorization request form, the treatment authorization request form having a plurality of fields, at least a portion of the plurality of fields corresponding to at least a portion of the plurality of elements;
associating the plurality of fields with the data set; and
merging the data set and the treatment authorization request form into an integrated file, at least a portion of the plurality of elements being represented on at least a portion of the plurality of fields.
